(19) United States
(12) Patent Application Publication
(10) Pub. No.: US 2005/0182026 A1
(43) Pub. Date: Aug. 18, 2005

(54) Use of phospholipids in peritoneal dialysis

(76) Inventors: Brian Andrew Hills, QUEENSLAND (AU); Derek Woodcock, Berkhampstead (GB); Jim Thompson, Coleshill (GB)

Correspondence Address: FISH & RICHARDSON PC, 225 FRANKLIN ST, BOSTON, MA, 02110, US

(21) Appl. No.: 10/501,184

(22) PCT Filed: Jan. 14, 2003
(86) PCT No.: PCT/GB03/00086

(30) Foreign Application Priority Data
Jan. 14, 2002 (GB) 0200704.5

(51) Int. Cl. A61K 31/685

(52) U.S. Cl. 514/78

(57) ABSTRACT

The efficiency of ultrafiltration in continuous ambulatory peritoneal dialysis (CAPD) is improved by administering a composition comprising at least one surface active phospholipid (SAPL) in powder form, especially as a mixture of phosphatidyl choline and phosphatidyl glycerol, into the peritoneal cavity before commencing CAPD or between CAPD sessions. The SAPL composition may be introduced during surgery to prepare a patient for CAPD and/or subsequently through the incision for the CAPD catheter, or through the catheter itself, between CAPD sessions when one batch of dialysis fluid has been removed and before a fresh batch is supplied.

This invention relates to the use of surface active phospholipids (SAPL) to improve the efficiency of ultrafiltration (UF) in patients on continuous ambulatory peritoneal dialysis (CAPD).

In 1985, Grahame et al (Perit. Dial. Bull. 1985; 5:109-111) identified surface-active phospholipids (SAPL) within the peritoneal cavities of patients on continuous ambulatory peritoneal dialysis (CAPD). This followed the earlier discovery of SAPL in the pleural cavity by Hills et al (J. Appl. Physiol. 1982; 53:463-469) and forming an oligolamellar lining which lubricates the pleural mesothelium. A similar lining has since been demonstrated reversibly bound (adsorbed) to peritoneal mesothelium; while the efficacy of adsorbed peritoneal SAPL to act as a boundary lubricant and release agent has been demonstrated by standard physical tests (Chen and Hills; Aust. N. Z. J. Surg. 2000; 70:443-447).

Grahame's discovery led to a somewhat tenuous link being established between any reduction in ultrafiltration (UF) in CAPD patients and an increasing loss of SAPL in their spent dialysate (Di Paolo et al; Perit. Dial. Bull. 1986; 6:44-45). This finding led to a spate of clinical trials in the late 1980s and early 1990s in which peritoneal surfactant was replenished in patients by spiking dialysis fluid with exogenous SAPL. The wide spectrum of outcomes ranged from several totally negative results to others where UF was increased. In the few studies where theory was discussed, the mechanism was generally attributed to a rather nebulous role for SAPL in eliminating a stagnant liquid layer adjacent to the mesothelium (Breborowicz et al; Perit. Dial. Bull. 1987; 7:6-9) although, in specific studies, such a fluid boundary layer has been dispelled as offering no significant resistance to mass transfer of solutes in PD (Flessner et al; Am. J. Physiol. 1985; 248:F413-424). Subsequently a study by Beavis et al (J. Am. Soc. Nephrol. 1993; 3:1954-1960) held that there is no relationhsip between dialysate phospholipid levels and the adequacy of UF, and that there was no support for a rationale for intraperitoneal phosphatidyl choline administration in CAPD patients with poor UF.

The present invention starts from the knowledge (Chen and Hills, above) that there is a lining of surface active phospholipid (SAPL) reversibly bound (adsorbed) to normal peritoneal mesothelium which acts as a boundary lubricant and release agent preserving mechanical integrity of this epithelial surface. The present invention is based on the finding that indigenous peritoneal SAPL is capable of imparting semipermeability to a surface to which it is adsorbed, leading to the conclusion that adsorbed SAPL imparts to peritoneal mesothelium the semi-permeability vital for UF and that any deficiency in SAPL can compromise UF.

The present invention is based on the use of powder compositions of phospholipids and liquid, semi-liquid or pasty compositions of phospholipids dispersed in a physiologically acceptable carrier to promote UF in CAPD patients by administering the compositions directly into the peritoneal cavity or by addition of the compositions to the dialysate used in CAPD.

SAPL powders as described in WO 99/51244 (Britannia) are easily administered into body cavities such as the peritoneum by simple “puffers” or other gas stream delivery devices, and the indicated SAPLs spread rapidly into inaccessible areas. Other suitable compositions are the liquid and paste SAPL compositions disclosed in U.S. Pat. No. 6,133,249 (Hills).

In one aspect the present invention provides a method of improving the efficiency or reducing deficiency of ultrafiltration in continuous ambulatory peritoneal dialysis which comprises administering a composition comprising at least one SAPL in powder form or dispersed or dissolved in a physiologically acceptable non-volatile carrier liquid into the peritoneal cavity before commencing CAPD or between CAPD sessions.

Thus the SAPL may be introduced during surgery to prepare a patient for CAPD; and/or subsequently through the incision for the CAPD catheter, or through the catheter itself, between CAPD sessions when one batch of dialysis fluid has been removed and before a fresh batch is supplied.

In another aspect the present invention provides a method of improving the efficiency or reducing deficiency of ultrafiltration in continuous ambulatory peritoneal dialysis which comprises administering a composition comprising at least one SAPL in powder form or dispersed or dissolved in a physiologically acceptable non-volatile carrier liquid (other than saline) into the dialysis fluid before commencing a CAPD session.

In this aspect the SAPL composition is mixed with the dialysis fluid and delivered with the dialysis fluid via the catheter provided for the fluid in a CAPD session.

In another aspect the present invention provides the use of at least one SAPL in powder form or dispersed or dissolved in a physiologically acceptable non-volatile carrier liquid (other than saline) to prepare a medicament for reducing improving the efficiency or reducing deficiency of ultrafiltration in continuous ambulatory peritoneal dialysis.

Examples of SAPLs which may be used in this invention include phosphatidylcholine (PC), in particular as diacyl phosphatidylcholines (DAPCs), e.g. dioleyl phosphatidylcholine (DOPC); distearyl phosphatidylcholine (DSPC) and dipalmnitoylphosphatidyl choline (DPPC). A spreading agent may be included which functions to reduce the melting point of a DAPC so that it rapidly spreads as a thin film at normal body temperature. Suitable spreading agents include phosphatidyl glycerols (PG); phosphatidyl ethanolamines (PE); phosphatidyl serines (PS) and phosphatidyl inositols (PI). Another useful spreading agent is chlorestyl palmitate (CP).

The above spreading agents, especially PG, are believed to enhance or potentiate the binding of the DAPC, especially the DPPC, to an epithelial surface. However compositions based on DPPC alone may sometimes be as effective as compositions based on DPPC/PG.

Also pastes prepared by dispersing coarse SAPL particles, for example around 10 μm in size, may be more effective than when using fine SAPL particles, such as around 5 μm in size. More generally, the powdered SAPL may have a particle size in the range of 0.5 to 100 μm, more suitably of 0.5 to 20 μm, preferably 0.5 to 10 μm.

Most suitably the dry SAPL composition is prepared from phosphatidylcholine (PC) and phosphatidyl glycerol (PG), but the invention is not limited solely to use of these lipids. Natural endogenous materials contain neutral lipids, fats, inorganic ions etc, all of which are integral to their form and function, and inclusion of these in formulations for use in the invention is not excluded. Preferred SAPL compositions are synthetic dipalmitoyl phosphatidylcholine (DPPC) co-precipitated from a common solvent system with PG in the weight ratio of 6:4 to 8:2, especially about 7:3. The composition is advantageously administered as a dry powder since it spreads extremely rapidly on water.

The phospholipids used in accordance with the invention have acyl substituents on the phosphatidyl groups. As in their natural counterparts, the acyl groups may comprise identical or different, saturated or unsaturated acyl radicals, generally C14-22, especially C16-20, acyl radicals. Thus the phospholipids may comprise, by way of acyl radicals, the saturated radicals palmitoyl C16:0 and stearoyl C18:0 and/or the unsaturated radicals oleoyls C18:1 and C18:2. Diacyl substitution is preferred and the phospholipids used in the compositions in accordance with the invention more particularly comprise two identical saturated acyl radicals, especially dipalmitoyl and distearoyl, or a mixture of phospholipids in which such radicals predominate, in particular mixtures in which dipalmitoyl is the major diacyl component. Thus PC and PG may be used may be used with the same diacylphosphatidyl profile as in PC and PG extracted from human or animal or vegetable sources, but if synthetic sources are used the dipalmitoyl component may predominate, as in the DPPC mentioned above.

As also mentioned above, the SAPL compositions are most preferably protein free, but in some circumstances the presence of proteins and adjuvants, especially naturally occurring materials from plant or animal sources, or synthetically derived, may be tolerated, especially proteins associated with PC and PG in vivo in conjunction with a dry powdered formulation for use in this invention. Especially apoprotein B marginally improves SAPL adsorption, and so may be useful if tolerated in SAPL compositions for human use.

DPPC can be prepared synthetically by acylation of glycerylphosphorylcholine using the method of Baer & Bachrea—Can. J. of Biochem. Physiol 1959, 37, page 953 and is available commercially from Sigma (London) Ltd. The PG may be prepared from egg phosphatidyl-choline by the methods of Comfurions et al, Biochem. Biophys Acta 1977, 488, pages 36 to 42; and Dawson, Biochem J. 1967, 102, pages 205 to 210, or from other phosphatidyl cholines, such as soy lecithin.

When co-precipitated with DPPC from a common solvent such as chloroform, PG forms with DPPC a fine powder which spreads rapidly over the surfaces of the airways and lungs. The most preferred composition of the invention contains DPPC and a phosphatidyl glycerol derived from egg phosphatidyl choline, which results in a mixture of C16, C18 (saturated and unsaturated) and C20 (unsaturated) acyl groups.

The SAPL compositions preferably used in accordance with the present invention are finely-divided, solid powders and are described in detail in our co-pending PCT applications WO 99/27920 and WO 00/30654, the whole contents of which are incorporated by reference. However in summary, our above applications indicate that an important feature of the SAPL compositions that are usable in the present invention is that they are in the form of a powder, that is, it is in solid form. The “dry” surfactant has a high surface activity.

When the SAPL is dispersed or dissolved in a carrier liquid, the carrier liquid is typically one which is substantially non-volatile or only sparingly volatile at body temperature. Suitable carriers include physiologically acceptable glycols, especially propylene glycol, polyethylene glycols and glycerol.

The SAPL may be dispersed in the carrier so as to form liquid, semi-liquid or pasty compositions. Semi-liquid or paste compositions are preferred.

Pastes can be prepared by simply dispersing a SAPL powder in the carrier, or when appropriate dissolving the SAPL(s) in heated carrier and allowing the SAPL(s) to precipitate as a powder on cooling, preferably at a loading that will form a paste. A thick paste of the SAPL and carrier is ideal to apply to open wounds to which it adheres well. It enables a much higher concentration of the SAPL to be applied to the incision site.

Propylene glycol is especially effective as a carrier because at room temperature SAPL may be dispersed in it as a paste, but at body temperature a mobile solution is formed. A paste of 400 mg/ml of DPPC in propylene glycol has given 93% protection against adhesions in surgical tests, as described in the experiments below.

Also polyethylene glycols may be prepared which are waxy solids at room temperature and liquids at body temperature, such as for example PEG 600.

Various dispersions of SAPLs in propylene glycol are described in U.S. Pat. No. 6,133,249, the entire contents of which are incorporated herein by reference. Similarly the powder compositions of WO 99/51244 may be dispersed in a carrier such as propylene glycol, and the entire disclosure of WO 99/51244 is also incorporated herein by reference.

In whichever form it is delivered, preferably the SAPL composition has two components. Suitably the first component of the SAPL comprises one or more compounds selected from the group consisting of diacyl phosphatidyl cholines. Examples of suitable diacyl phosphatidyl cholines (DAPCs), are dioleyl phosphatidyl choline (DOPC); distearyl phosphatidyl choline (DSPC) and dipalmitoyl phosphatidyl choline (DPPC). Most preferably, the first component is DPPC.

The second component may comprise one or more compounds selected from the group consisting of phosphatidyl glycerols (PG); phosphatidyl ethanolamines (PE); phosphatidyl serines (PS); phosphatidyl inositols (PI) and chlorestyl palmitate (CP).

Phosphatidyl glycerol (PG) is a preferred second component. PG is also a preferred second component because of its ability to form with the first component, especially PC and particularly DPPC, a very finely-divided, dry powder dispersion in air.

The composition advantageously comprises a diacyl phosphatidyl choline and a phosphatidyl glycerol. The phosphatidyl glycerol is advantageously a diacyl phosphatidyl glycerol. The acyl groups of the phosphatidyl glycerol, which may be the same or different, are advantageously each fatty acid acyl groups which may have from 14 to 22 carbon atoms. In practice, the phosphatidyl glycerol component may be a mixture of phosphatidyl glycerols containing different acyl groups. The phosphatidyl glycerol is expediently obtained by synthesis from purified lecithin, and the composition of the acyl substituents is then dependent on the source of the lecithin used as the raw material. It is preferred for at least a proportion of the fatty acid acyl groups of the phosphatidyl glycerol to be unsaturated fatty acid residues, for example, mono- or di-unsaturated C18 or C20 fatty acid residues.

Preferred acyl substituents in the phosphatidyl glycerol component are palmitoyl, oleoyl, linoleoyl, linolenoyl and arachidonoyl. The medicament preferably comprises dipalmitoyl phosphatidyl choline and phosphatidyl glycerol, with the phosphatidyl moiety of the phosphatidyl glycerol advantageously being obtainable from the phosphatidyl moiety of egg lecithin.

The compositions are administered preferably in a dry, finely-divided state, using a delivery device such as described in our above co-pending applications, or by directly introducing the aerosolised powder, e.g. by a tube which may be coated to aid transport of SAPL, into the peritoneal cavity.

While not wishing to be limited to the following theory it is believed that, when absorbed (reversibly bound) to the peritoneal mesothelium, SAPL provides a semi-permeable membrane by which the desired dialsysis is implemented. The predicated deficiency of SAPL which contributes to poor UF leads to a deficiency in this absorbed semi-permeable lining. This situation may be corrected by administering exogenous SAPL, advanatgeously in a form which displays two properties. First it spreads rapidly over the surface of the incumbent fluid for widespread distribution throughout the peritoneal cavity. Secondly, it then absorbs to the epithelial surface to repair/fortify the semi-permeable barrier comprising similar material.

It is highly desirable that the SAPL should not break down quickly at the surgical site in the body. One of the factors which will reduce the life of a lining or coating of SAPL will be the presence of enzymes, such as phospholipase A, capable of digesting DPPC and/or PG. Such enzymes only attack the laevorotatory (L) form, which constitutes the naturally occurring form. Therefore, it may be preferable to use the dextrorotatory (D) form of the SAPL(s) or at least a racemic mixture, which is obtained by synthetic routes.

The compositions may also include preservatives where appropriate, such as fungicides, bactericides and anti-oxidants.

The present invention is supported by the following experimental work.

Introduction

It has been previously demonstrated that there is a lining of surface active phospholipid (SAPL) reversibly bound (adsorbed) to normal peritoneal mesothelium which acts as a boundary lubricant and release agent preserving mechanical integrity of this epithelial surface. In reviewing clinical trials of the use of SAPL (alias “surfactant”) to restore ultrafiltration (UF) in patients on peritoneal dialysis (PD), we have speculated that the SAPL lining might also be imparting the semi-permeability vital for UF.

In evaluating this hypothesis, SAPL harvested from the spent dialysate of 5 patients with normal UF has been deposited on to a porous inert medium and the resulting 7 ‘membranes’ clamped in an Ussing chamber used as an osmometer. In every ‘membrane’ a clinical concentration of glucose (2.5%) was able to induce a statistically significant osmotic pressure when dialysed against saline. This proves that human peritoneal SAPL has the physical capability to impart semi-permeability when adsorbed to a surface. This could also explain the high permeability of the natural membrane to lipophilic substances in PD.

We have also demonstrated how synthetic SAPL in the form of dipalmitoylphosphatidylcholine (DPPC) and its admixture with phosphatidyl glycerol (pumactant) imparts greater osmotic pressure and does so in proportion to the glucose gradient. Both pumactant and DPPC in various physical forms have been widely used for two decades with complete safety in the treatment of the respiratory distress syndrome in newborns. As a very fine powder, pumactant offers a potential role in restoring UF if applied during the interdialytic interval.

The question of formulation of exogenous SAPL in restoring ultrafiltration is discussed as a complex physico-chemical compromise between the higher surface activity of saturated PC and its lower solubility in water.

Materials and Methods

Principle

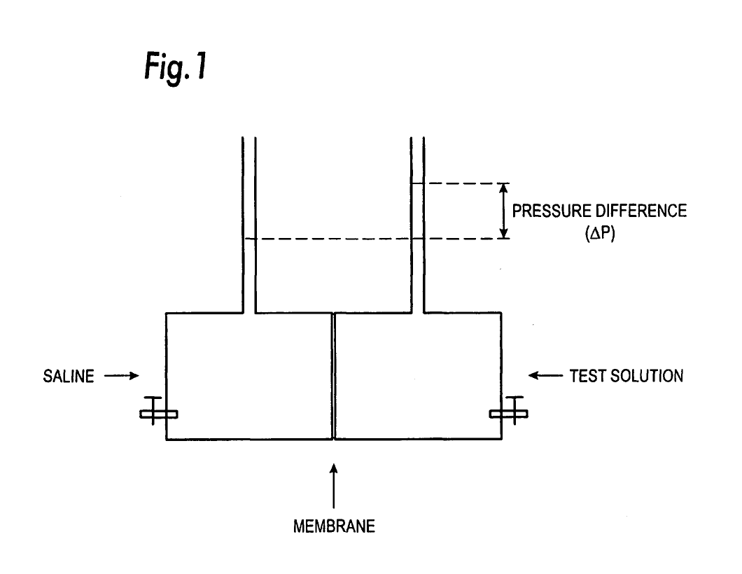

The mechanical base for ‘the membrane’ is a fine-pore filter paper proven to be totally permeable to glucose, urea and physiologically relevant ions. SAPL is then deposited as a thin coating and the resulting membrane clamped between the two compartments of an Ussing chamber to form an osmometer. Any osmotic pressure (ΔP) generated between the compartments is measured as the difference in hydrostatic pressure needed to balance ΔP and stop further osmosis—see FIG. 1. The SAPL is derived from spent dialysate from CAPD patients with normal UF and compared with synthetic surfactants envisaged as possible sources of replenishment of indigenous SAPL where UF is inadequate. The driving force for generating an osmotic pressure is provided by glucose in concentration gradients used clinically to induce and control UF in CAPD.

Materials

The synthetic surface-active phopholipid (SAPL) was either dipalmitoyl phosphatidylcholine (DPPC) purchased from Lipoid GmbH (Ludwigshafen, Germany) or pumactant provided by Britannia Pharmaceuticals Ltd (Redhill, UK). Human peritoneal SAPL was extracted from the spent dialysate of patients exhibiting normal UF using the Folch method (J. Biol. Chem. 1957; 226:497-509). All chemical reagents (chloroform, methanol and acetone) were at least AR grade and purchased from AJAX Chemicals (Auburn, NSW, Australia) or BDH Laboratory Supplies (Poole, UK). Saline and Dianeal-2 dialysis fluids with glucose concentrations of 1.5%, 2.5% and 4.25% (Baxter Healthcare, Old Toongabbie, NSW, Australia) provided the concentration gradients for generating osmotic pressure. Dialysis fluid with a glucose concentration of 3.4% was made by proportionally mixing two different dialysis fluids (with glucose concentrations of 2.5%; and 4.25%).

Methods

SAPL membranes were made by applying equal volumes of SAPL in chloroform solution on to both sides of a filter paper (0.2 μm, white nylon, Millipore Corporation, Bedford, USA). Osmotic pressure was measured by clamping the SAPL membranes between the two compartments of an Ussing chamber (Jim's Instrument Manufacturing, Inc., Iowa, USA). Osmotic pressure was measured as the difference in hydrostatic pressure of the compartments needed to stop further water transmission across the membrane. The total capacity and contact area of chambers are approximately 0.7 ml and 0.44 cm². SAPL (2.36 mg of DPPC, pumactant or human peritoneal SAPL) and 3.78 mg SAPL (DPPC or pumactant) were used for different experiments. Two vertical tubes with inner diameters of 1.2 mm were connected to the side, of each for measuring osmotic pressure. In the experiments, the left compartment was always filled with saline and the right side with test solution (Dianeal-2 dialysis fluids with different glucose concentrations). The device is illustrated in FIG. 1. At the beginning of the experiment the fluid heights indicating pressure were set the same on both sides of the membrane. The whole device was kept at 37° C. in a water bath and the fluid heights indicating pressure difference were measured and recorded until there was no further movement of fluid. At the end of each experiment the osmotic pressure was recorded as the difference in heights between the two fluid columns. The mean and S.E.M. were calculated for every group of data and the one-way ANOVA test was used for statistical analysis.

The whole study was divided into five sections:

Section I:

Measurement of osmotic pressure produced by dialysing saline against Dianeal-2 dialysis fluids with 2.5% glucose concentrations against DPPC (2.36 mg per preparation) membrane (N=8).

Section II:

Measurement of osmotic pressure produced by dialysing saline against Dianeal-2 dialysis fluid with 2.5% glucose concentration against pumactant (2.36 mg per preparation) membrane (N=8).

Section III:

Measurement of osmotic pressure produced by dialysing saline against Dianeal-2 dialysis fluid with 2.5% glucose concentrations using extracted human peritoneal SAPL (2.36 mg per preparation) membrane (N=7).

Section IV:

Measurement of osmotic pressure produced by dialysing saline against Dianeal-2 dialysis fluids with different glucose concentrations (1.5%, 2.5%, 3.4% and 4.25%) against DPPC (3.78 mg per preparation) membrane (N=8).

Section V:

Measurement of osmotic pressure produced by dialysing saline against Dianeal-2 dialysis fluid with different glucose concentrations (1-5%, 2.5%, 3.4% and 4.25%) using pumactant (3.78 mg per preparation) membrane (N=8).

Results

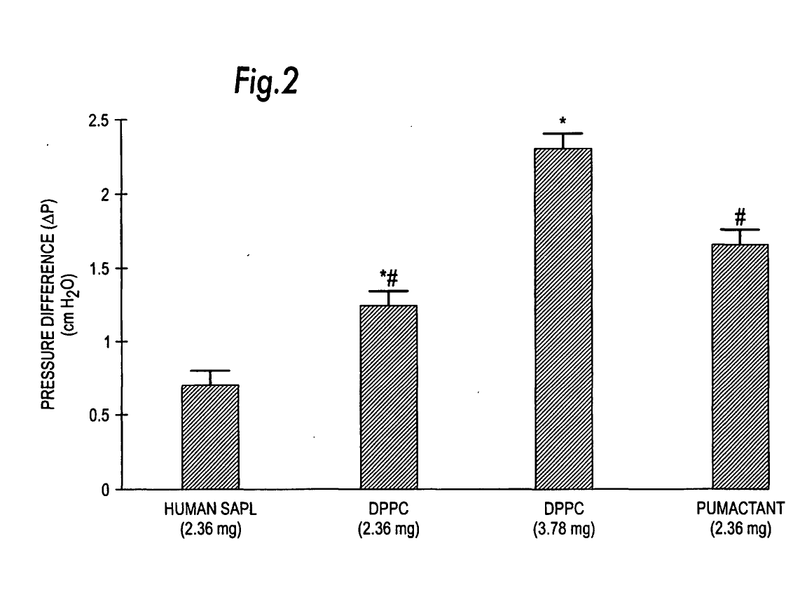

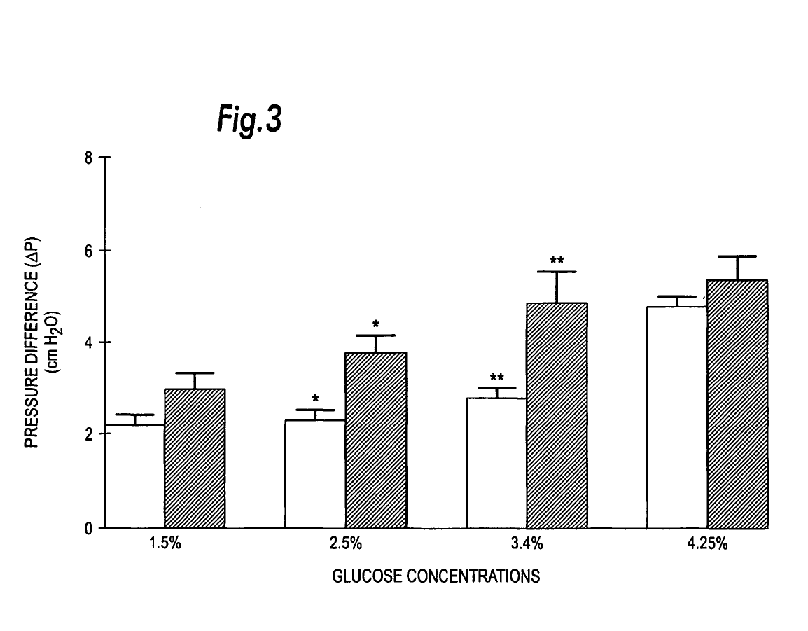

In all experiments an osmotic pressure was generated by dialysing any hypertonic dialysate against saline. The results from Sections I, II and III are given in FIG. 2 while those from Sections IV and V are compared in FIG. 3. The features of these results can be listed as follows:

1. In 7 runs, each using the pooled SAPL harvested from 5 exchanges, an osmotic pressure was always generated by Dianeal-2.

2. Synthetic SAPL was more effective than indigenous peritoneal SAPL with pumactant more effective than DPPC at the same (2.36 mg) thickness—see FIG. 2.

3. Thicker membranes (3.78 mg DPPC) were more effective than thinner membranes (2.36 mg DPPC),—see FIG. 2.

4. For the same membrane thickness and composition, the osmotic pressure increased with the glucose driving force for osmosis—see FIG. 3—as predicted by the van't Hoff equation governing osmosis.

5. Pumactant was more effective than pure DPPC at each glucose concentration. At glucose concentrations of 2.5% and 3.4%, pumactant membranes generated statistically significant higher osmotic pressures than DPPC membranes (p<0.05).

Discussion

Although the results of this study show convincingly that human peritoneal SAPL imparts semi-permeability to an inert porous base, it does not prove conclusively that it necessarily does the same to peritoneal mesothelium in vivo.

However, there are many factors which support this hypothesis. Firstly we have previously demonstrated by epifluorescence microscopy that there is a lining of SAPL adsorbed to parietal peritoneum which is probably oligolamellar in nature, resembling similar linings adsorbed to pleural mesothelium. Secondly, oligolamellar layers of SAPL in the form of liposomes have long been known to be semi-permeable to such low-molecular weight solutes as NaCl. Thirdly, there is the evidence from clinical trials that there is a association between reduction of UF in PD and loss of SAPL in dialysate. It could be argued that the quantity recovered from spent dialysate does not necessarily reflect the amount of SAPL adsorbed to parietal mesothelium which surface accounts for 85% of dialysis. However we have demonstrated that exogenous SAPL in the form of radiolabelled DPPC does indeed adsorb to parietal mesothelium. This raises the issue of whether the administration of exogenous SAPL should be employed for the restoration of UF in patients who have lost that capability and what insight the adsorption theory may offer in the formulation of exogenous SAPL for this purpose. In addition, an SAPL barrier would help to explain why the peritoneal membrane is an order of magnitude more permeable to lipid-soluble substances than to other solutes.

In attempting to review the many clinical trials of SAPL in improving UF, the most frustrating aspect was the lack of physico-chemical information on the widely diverse range of formulations which have been tested. Adsorption is a specialised branch of physical chemistry in which the Langmuir isotherm relates the quantity of a substance adsorbed to its concentration in the adjacent fluid phase. The two parameters most desirable for high adsorption of any substance to a solid surface are high surface activity and high solubility in the adjacent phase—dialysate in the case of PD. Hence we selected DPPC as one of our exogenous surfactants because it is generally regarded as the most surface-active phospholipid. This did indeed display better semi-permeability when used in the Ussing chamber as displayed in FIG. 2. Unfortunately it is highly insoluble in water as demonstrated by a critical micelle concentration as low as 5×10⁻¹⁰ Molar (20). In order to circumvent this problem, and largely to improve spreading, DPPC has been used as an intimate mixture with PG (pumactant) in the use of surfactant in treating neonates born with the respiratory distress syndrome. Hence it could be fortuitous that not only is this mixture easier to dispense in aqueous fluids, but it has demonstrated the best results in its ability to impart semi-permeability—see FIG. 3. This is encouraging because, when applied as a fine dry powder to the peritoneum, it offered excellent results in preventing surgical adhesions. It would need to be adsorbed strongly to peritoneal mesothelium in order to act as an effective boundary lubricant and release (anti-stick) agent protecting the peritoneum. This raises the possibility of using the interdialytic interval as an opportunity to replenish SAPL, adsorbed to peritoneal mesothelium and hence restore ultrafiltration—whether prescribed as a dry powder (e.g. pumactant) or dispensed in dialysate.

In conclusion, there is good evidence that adsorbed surface active phospholipid is providing the semi-permeability of the mesothelium vital for ultrafiltration, this mechanism offering a new physico-chemical approach to the formulation of SAPL for restoring ultrafiltration as set out in the present invention.

1. A method of improving the efficiency or reducing deficiency of ultrafiltration in continuous ambulatory peritoneal dialsysis which comprises administering a composition comprising at least one SAPL in powder form or dispersed or dissolved in a physiologically acceptable non-volatile carrier liquid into the peritoneal cavity before commencing CAPD or between CAPD sessions.

2. A method of improving the efficiency or reducing deficiency of ultrafiltration in continuous ambulatory peritoneal dialysis which comprises administering a composition comprising at least one SAPL in powder form or dispersed or dissolved in a physiologically acceptable non-volatile carrier liquid (other than saline) into the dialysis fluid before commencing a CAPD session.

3. Use of at least one SAPL in powder form or dispersed or dissolved in a physiologically acceptable non-volatile carrier liquid (other than saline) to prepare a medicament for reducing improving the efficiency or reducing deficiency of ultrafiltration in continuous ambulatory peritoneal dialysis.

4. Use or method according to claim 1 in the SAPL is selected from diacyl phosphatidylcholines (DAPCs), such as dioleyl phosphatidylcholine (DOPC); distearyl phosphatidylcholine (DSPC) and dipalmitoyl phosphatidylcholine (DPPC).

5. Use or method according to claim 4 in which the SAPL composition further includes a spreading agent such as a phosphatidyl glycerol (PG), phosphatidyl ethanolamine (PE), phosphatidyl serine (PS), phosphatidyl inositol (PI) or chlorestyl palmitate (CP).

6. Use or method according to claim 1 in which the SAPL composition is a mixture of phosphatidylcholine (PC) and phosphatidyl glycerol (PG).

7. Use or method according to claim 6 in which the SAPL composition is a mixture of dipalmitoyl phosphatidylcholine (DPPC), or a phosphatidylcholine blend (PC) which is predominantly dipalmitoyl phosphatidylcholine (DPPC), and phosphatidyl glycerol (PG).

8. Use or method according to claim 1 in which the carrier is glycerol, propylene glycol, or a polyethylene glycol.

9. Use or method according to claim 8 in which the carrier is propylene glycol.

10. Use or method according to claim 1 in which the SAPL/carrier is in the form of a paste.

11. Use or method according to claim 2 in the SAPL is selected from diacyl phosphatidylcholines (DAPCs), such as dioleyl phosphatidylcholine (DOPC); distearyl phosphatidylcholine (DSPC) and dipalmitoyl phosphatidylcholine (DPPC).

12. Use or method according to claim 3 in the SAPL is selected from diacyl phosphatidylcholines (DAPCs), such as dioleyl phosphatidylcholine (DOPC); distearyl phosphatidylcholine (DSPC) and dipalmitoyl phosphatidylcholine (DPPC).

13. Use or method according to claim 11 in which the SAPL composition further includes a spreading agent such as a phosphatidyl glycerol (PG), phosphatidyl ethanolamine (PE), phosphatidyl serine (PS), phosphatidyl inositol (PI) or chlorestyl palmitate (CP).

14. Use or method according to claim 12 in which the SAPL composition further includes a spreading agent such as a phosphatidyl glycerol (PG), phosphatidyl ethanolamine (PE), phosphatidyl serine (PS), phosphatidyl inositol (PI) or chlorestyl palmitate (CP).

15. Use or method according to claim 2 in which the SAPL composition is a mixture of phosphatidylcholine (PC) and phosphatidyl glycerol (PG).

16. Use or method according to claim 3 in which the SAPL composition is a mixture of phosphatidylcholine (PC) and phosphatidyl glycerol (PG).

17. Use or method according to claim 15 in which the SAPL composition is a mixture of dipalmitoyl phosphatidylcholine (DPPC), or a phosphatidylcholine blend (PC) which is predominantly dipalmitoyl phosphatidylcholine (DPPC), and phosphatidyl glycerol (PG).

18. Use or method according to claim 16 in which the SAPL composition is a mixture of dipalmitoyl phosphatidylcholine (DPPC), or a phosphatidylcholine blend (PC) which is predominantly dipalmitoyl phosphatidylcholine (DPPC), and phosphatidyl glycerol (PG).

19. Use or method according to claim 2 in which the carrier is glycerol, propylene glycol, or a polyethylene glycol.

20. Use or method according to claim 3 in which the carrier is glycerol, propylene glycol, or a polyethylene glycol.
